(19) United States
(12) Patent Application Publication
(10) Pub. No.: US 2018/0085024 A1
(43) Pub. Date: Mar. 29, 2018

(54) SYSTEM AND METHOD FOR MAGNETIC RESONANCE ANGIOGRAPHY USING HYPERPOLARIZED FLUID

(71) Applicant: The General Hospital Corporation, Boston, MA (US)

(72) Inventors: Matthew S. Rosen, Somerville, MA (US); Mathieu Sarracanie, Somerville, MA (US); Najat Salameh, Somerville, MA (US)

(21) Appl. No.: 15/562,547

(22) PCT Filed: Apr. 1, 2016
(86) PCT No.: PCT/US16/25486

§ 371 Date: Sep. 28, 2017

Related U.S. Application Data
(60) Provisional application No. 62/141,529, filed on Apr. 1, 2015.

(51) Int. Cl. A61B 5/055; G01R 33/38; G01R 33/44; G01R 33/56; G01V 3/14; G01R 33/561

(52) U.S. Cl. CPC A61B 5/055; G01R 33/3806; G01R 33/445; G01R 33/381; G01V 3/14; G01R 33/5614; G01R 33/5601

(57) ABSTRACT

Systems and methods are provided for performing a medical imaging. The system includes a main magnet system configured to generate a main static magnetic field about at least a region of interest (ROD of a subject arranged in the main magnet system. The system also includes at least one gradient coil configured to establish at least one magnetic gradient field with respect to the main static magnetic field, The system also includes a radio frequency (RF) system configured to deliver excitation pulses to the subject. The system also includes a polarization magnet system configured to generate a polarization static magnetic field in an at least partially shielded region away from the main magnet system, the polarization magnet system configured to at least partially pre-polarize a blood substitute to be injected to the subject.

### CROSS-REFERENCE TO RELATED APPLICATIONS

This application is based on, claims priority to, and incorporates herein by reference, U.S. Provisional Application Ser. No. 62/141,529, filed Apr. 1, 2015, and entitled “HYPERPOLARIZED WATER FOR MRI.”

### STATEMENT REGARDING FEDERALLY SPONSORED RESEARCH

This disclosure was made with government support under W81XWH-11-2-076 awarded by the Department of Defense. The government has certain rights in the disclosure.

### FIELD

The present disclosure relates to systems and methods for the disclosure is magnetic resonance imaging (MRI). More particularly, the present disclosure provides systems and methods for imaging hyperpolarized fluid using MRI system.

### BACKGROUND

When a substance such as human tissue is subjected to a uniform magnetic field (polarizing field B₀), the individual magnetic moments of the excited nuclei in the tissue attempt to align with this polarizing field, and precess about it in random order at their characteristic Larmor frequency. If the substance, or tissue, is subjected to a magnetic field (excitation field B₁) which is in the x-y plane and which is near the Larmor frequency, the net aligned moment, M_{z}, may be rotated, or “tipped”, into the x-y plane to produce a net transverse magnetic moment Mₜ. A signal is emitted by the excited nuclei or “spins”, after the excitation signal B₁ is terminated, and this signal may be received and processed to form an image.

When utilizing these “MR” signals to produce images, magnetic field gradients (Gₓ, G_{y}, and G_{z}) are employed. Typically, the region to be imaged is scanned by a sequence of measurement cycles in which these gradients vary according to the particular localization method being used. The resulting set of received MR signals are digitized and processed to reconstruct the image using one of many well known reconstruction techniques.

Acute reperfusion therapies have changed ischemic stroke care, but treatments are limited because of a short therapeutic window owing to the risk of reperfusion injury and hemorrhage. Detection of early and mild blood-brain barrier (BBB) disruption is an unmet need in acute stroke diagnosis. Although contrast from relaxation-based MRI contrast agents such as Gd-DTPA is correlated with hemorrhagic transformation of an infarct, it is not sensitive enough to probe more mild BBB disruption.

Over the last decade, researchers have put significant efforts in developing new probes for molecular imaging where contrast agents would target only specific cells and/or regions. However, when using contrast-enhanced MRI in oncology and abdominal imaging, one main challenge is to determine the potential toxicity of the contrast agent. Thus, further developments are necessary to meet clinical needs.

### SUMMARY

The present disclosure overcomes the aforementioned drawbacks by providing a MRI system and method for imaging in conjunction with hyperpolarized fluid. The MRI system generates a polarization static magnetic field in an at least partially shielded region away from the main magnet system and at least partially pre-polarizes a blood substitute to be injected.

In accordance with one aspect of the disclosure, a magnetic resonance imaging (MRI) system is disclosed that is configured to perform an imaging process of a subject having received hyperpolarized fluid. The system includes a main magnet system configured to generate a main static magnetic field about at least a region of interest (ROI) of a subject arranged in the main magnet system. The system also includes at least one gradient coil configured to establish at least one magnetic gradient field with respect to the main static magnetic field. The system also includes a radio frequency (RF) system configured to deliver excitation pulses to the subject. The system also includes a polarization magnet system configured to generate a polarization static magnetic field in an at least partially shielded region away from the main magnet system, the polarization magnet system configured to at least partially pre-polarize a blood substitute to be injected to the subject.

In accordance with another aspect of the disclosure, a method is provided for performing a medical imaging process. The method includes providing a main magnet system that generates a main static magnetic field about at least a region of interest (ROI) of a subject arranged in the main magnet system. The method includes providing a polarization magnet system that generates a polarization static magnetic field in an at least partially shielded region away from the main magnet system. The method includes pre-polarizing a blood substitute to be injected to the subject using the polarization magnet system. The method includes performing an magnetic resonance imaging (MRI) process to acquire data from the subject in the main magnet system. The method further includes reconstructing the data to generate a report indicating a spatial distribution of the pre-polarized blood substitute in the subject.

In a third aspect of the disclosure, a MRI system is provided. The MRI system includes a main magnet system configured to generate a main magnetic field about at least a region of interest (ROI) of a subject arranged in the MRI system. The MRI system includes a polarization magnet system configured to generate a polarization static magnetic field in an at least partially shielded region away from the main magnet system, the polarization magnet system configured to at least partially pre-polarize a fluid comprising fluorine. The MRI system includes a radio frequency (RF) system configured to deliver excitation pulses to the subject having received the pre-polarize a fluid comprising fluorine, wherein the excitation pulses comprises at least one embedded electron paramagnetic resonance (EPR) pulse. The MRI system includes a controller configured to at least partially manipulate a strength of the pre-polarization by controlling a flow rate of the pre-polarized fluid.

The foregoing and other advantages of the disclosure will appear from the following description.

### BRIEF DESCRIPTION OF THE DRAWINGS

FIG. 1 is a block diagram of an MRI system.

FIG. 2 is a block diagram of an RF system of an MRI system.

FIG. 3 is a picture of a low-field MRI (lfMRI) system in accordance with the present disclosure.

FIG. 4a is a block diagram of an MRI system in accordance with the present disclosure.

FIG. 4b is a zoomed in view of the MRI system in accordance with the present disclosure.

FIG. 5a illustrates an example method in accordance with the present disclosure.

FIG. 5b illustrates additional acts that may be implemented in accordance with the present disclosure.

FIG. 6a shows a phantom image with 5 averages without hyperpolarized fluid.

FIG. 6b shows a phantom image with 5 averages and hyperpolarized fluid.

FIG. 6c shows a reference scan with 20 averages where the input and output are clearly visible.

FIG. 6d shows the overlay of FIG. 6b on FIG. 6c.

FIG. 7 shows a shielding box in accordance with the instant disclosure.

### DETAILED DESCRIPTION

Contrast-enhanced MRI relies on changing the relaxivity of tissues, either by reducing T1 allowing an enhanced signal on T1-weighted images, or by reducing T2* and thus relying on negative contrast on T2/T2* weighted images to track the uptake of the contrast agent. At ultra-low field (ULF), T1 is significantly shorter compared to high field, and the ULF regime is generally immune to T2* effects suggesting that conventional contrast agents will not be of tremendous utility for ULF imaging. The disclosure provides a new approach in ULF imaging by using hyperpolarized fluid as contrast medium. For example, hyperpolarized saline and artificial blood substitute may be used. Saline is biologically safe, and with the use of a small, strong pre-polarizing permanent magnet, enables contrast-enhanced MRI at 0.0065 T.

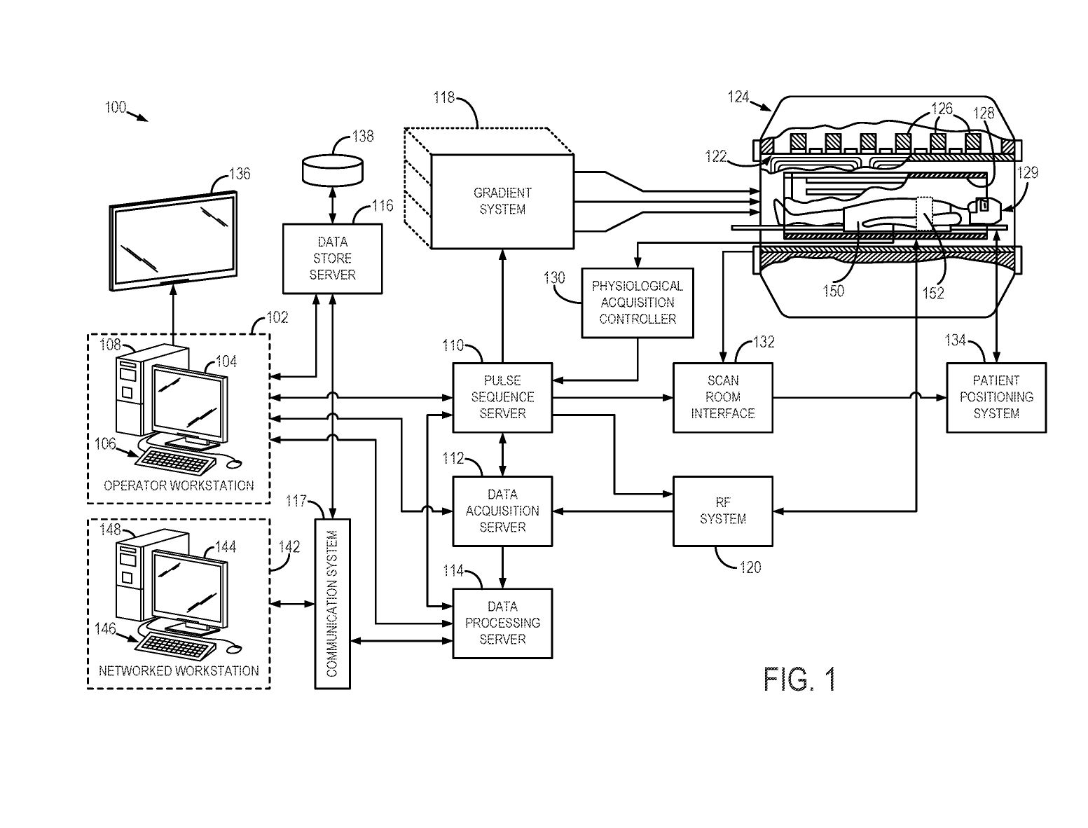

Referring particularly now to FIG. 1, an example of a magnetic resonance imaging (MRI) system 100 is illustrated. The MRI system 100 includes an operator workstation 102, which will typically include a display 104, one or more input devices 106, such as a keyboard and mouse, and a processor 108. The processor 108 may include a commercially available programmable machine running a commercially available operating system. The operator workstation 102 provides the operator interface that enables scan prescriptions to be entered into the MRI system 100. In general, the operator workstation 102 may be coupled to four servers: a pulse sequence server 110; a data acquisition server 112; a data processing server 114; and a data store server 116. The operator workstation 102 and each server 110, 112, 114, and 116 are connected to communicate with each other. For example, the servers 110, 112, 114, and 116 may be connected via a communication system 117, which may include any suitable network connection, whether wired, wireless, or a combination of both. As an example, the communication system 117 may include both proprietary or dedicated networks, as well as open networks, such as the internet.

The pulse sequence server 110 functions in response to instructions downloaded from the operator workstation 102 to operate a gradient system 118 and a radiofrequency (“RF”) system 120. Gradient waveforms necessary to perform the prescribed scan are produced and applied to the gradient system 118, which excites gradient coils in an assembly 122 to produce the magnetic field gradients Gₓ, G_{y}, and G_{z} used for position encoding magnetic resonance signals. The gradient coil assembly 122 forms part of a magnet assembly 124 that includes a polarizing magnet 126 and a whole-body RF coil 128 and/or local coil, such as a head coil 129.

The MRI system 100 may specify a region of interest (ROI) 152 in the subject 150 by manipulating the gradient system 118 and the RF system 120. The MRI system 100 may apply additional transmitting or receiving coils to image an ROI 152 in the subject 150.

RF waveforms are applied by the RF system 120 to the RF coil 128, or a separate local coil, such as the head coil 129, in order to perform the prescribed magnetic resonance pulse sequence. Responsive magnetic resonance signals detected by the RF coil 128, or a separate local coil, such as the head coil 129, are received by the RF system 120, where they are amplified, demodulated, filtered, and digitized under direction of commands produced by the pulse sequence server 110. The RF system 120 includes an RF transmitter for producing a wide variety of RF pulses used in MRI pulse sequences. The RF transmitter is responsive to the scan prescription and direction from the pulse sequence server 110 to produce RF pulses of the desired frequency, phase, and pulse amplitude waveform. The generated RF pulses may be applied to the whole-body RF coil 128 or to one or more local coils or coil arrays, such as the head coil 129.

The RF system 120 also includes one or more RF receiver channels. Each RF receiver channel includes an RF preamplifier that amplifies the magnetic resonance signal received by the coil 128/129 to which it is connected, and a detector that detects and digitizes the I and Q quadrature components of the received magnetic resonance signal. The magnitude of the received magnetic resonance signal may, therefore, be determined at any sampled point by the square root of the sum of the squares of the I and Q components:

M=\sqrt{I²+Q²} (1):

and the phase of the received magnetic resonance signal may also be determined according to the following relationship:

$$ϕ = {tan}^{- 1} ( \frac{Q}{I} ) . \qquad ( 2 )$$

The pulse sequence server 110 also optionally receives patient data from a physiological acquisition controller 130. By way of example, the physiological acquisition controller 130 may receive signals from a number of different sensors connected to the patient, such as electrocardiograph (“ECG”) signals from electrodes, or respiratory signals from a respiratory bellows or other respiratory monitoring device. Such signals are typically used by the pulse sequence server 110 to synchronize, or “gate,” the performance of the scan with the subject's heart beat or respiration.

The pulse sequence server 110 also connects to a scan room interface circuit 132 that receives signals from various sensors associated with the condition of the patient and the magnet system. It is also through the scan room interface circuit 132 that a patient positioning system 134 receives commands to move the patient to desired positions during the scan.

The digitized magnetic resonance signal samples produced by the RF system 120 are received by the data acquisition server 112. The data acquisition server 112 operates in response to instructions downloaded from the operator workstation 102 to receive the real-time magnetic resonance data and provide buffer storage, such that no data is lost by data overrun. In some scans, the data acquisition server 112 does little more than pass the acquired magnetic resonance data to the data processor server 114. However, in scans that require information derived from acquired magnetic resonance data to control the further performance of the scan, the data acquisition server 112 is programmed to produce such information and convey it to the pulse sequence server 110. For example, during prescans, magnetic resonance data is acquired and used to calibrate the pulse sequence performed by the pulse sequence server 110. As another example, navigator signals may be acquired and used to adjust the operating parameters of the RF system 120 or the gradient system 118, or to control the view order in which k-space is sampled. In still another example, the data acquisition server 112 may also be employed to process magnetic resonance signals used to detect the arrival of a contrast agent in a magnetic resonance angiography (MRA) scan. By way of example, the data acquisition server 112 acquires magnetic resonance data and processes it in real-time to produce information that is used to control the scan.

Here, the data acquisition server 112 may further include a computer system programmed to control the at least one gradient coil and the RF system to perform a MRI pulse sequence. The computer system may be further programmed to acquire data corresponding to signals from the subject having received at least partially pre-polarized blood substitute from the polarization magnet system. Moreover, the computer system may be further programmed to reconstruct, from the data, at least one anatomical image of the subject and spatially distributed pre-polarized blood substitute within the subject relative to the anatomical image.

The data processing server 114 receives magnetic resonance data from the data acquisition server 112 and processes it in accordance with instructions downloaded from the operator workstation 102. Such processing may, for example, include one or more of the following: reconstructing two-dimensional or three-dimensional images by performing a Fourier transformation of raw k-space data; performing other image reconstruction algorithms, such as iterative or backprojection reconstruction algorithms; applying filters to raw k-space data or to reconstructed images; generating functional magnetic resonance images; calculating motion or flow images; and so on.

Images reconstructed by the data processing server 114 are conveyed back to the operator workstation 102 where they are stored. Real-time images are stored in a data base memory cache (not shown in FIG. 1), from which they may be output to operator display 112 or a display 136 that is located near the magnet assembly 124 for use by attending physicians. Batch mode images or selected real time images are stored in a host database on disc storage 138. When such images have been reconstructed and transferred to storage, the data processing server 114 notifies the data store server 116 on the operator workstation 102. The operator workstation 102 may be used by an operator to archive the images, produce films, or send the images via a network to other facilities.

The MRI system 100 may also include one or more networked workstations 142. By way of example, a networked workstation 142 may include a display 144; one or more input devices 146, such as a keyboard and mouse; and a processor 148. The networked workstation 142 may be located within the same facility as the operator workstation 102, or in a different facility, such as a different healthcare institution or clinic.

The networked workstation 142, whether within the same facility or in a different facility as the operator workstation 102, may gain remote access to the data processing server 114 or data store server 116 via the communication system 117. Accordingly, multiple networked workstations 142 may have access to the data processing server 114 and the data store server 116. In this manner, magnetic resonance data, reconstructed images, or other data may exchanged between the data processing server 114 or the data store server 116 and the networked workstations 142, such that the data or images may be remotely processed by a networked workstation 142. This data may be exchanged in any suitable format, such as in accordance with the transmission control protocol (TCP), the internet protocol (IP), or other known or suitable protocols.

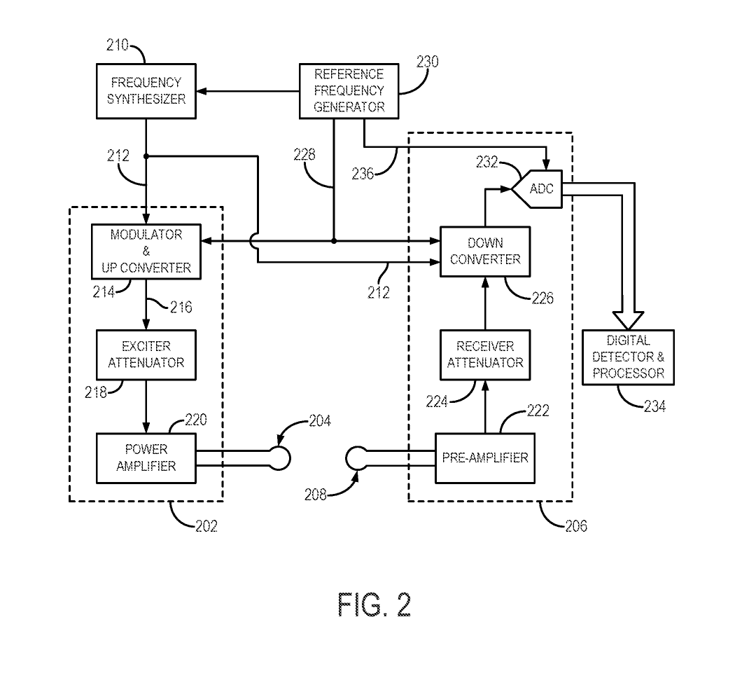

With reference to FIG. 2, the RF system 120 of FIG. 1 will be further described. The RF system 120 includes a transmission channel 202 that produces a prescribed RF excitation field. The base, or carder, frequency of this RF excitation field is produced under control of a frequency synthesizer 210 that receives a set of digital signals from the pulse sequence server 110. These digital signals indicate the frequency and phase of the RF carrier signal produced at an output 212. The RF carrier is applied to a modulator and up converter 214 where its amplitude is modulated in response to a signal, R(t), also received from the pulse sequence server 110. The signal, R(t), defines the envelope of the RF excitation pulse to be produced and is produced by sequentially reading out a series of stored digital values. These stored digital values may be changed to enable any desired RF pulse envelope to be produced.

The magnitude of the RF excitation pulse produced at output 216 is attenuated by an exciter attenuator circuit 218 that receives a digital command from the pulse sequence server 110. The attenuated RF excitation pulses are then applied to a power amplifier 220 that drives the RF transmission coil 204.

The MR signal produced by the subject is picked up by the RF receiver coil 208 and applied through a preamplifier 222 to the input of a receiver attenuator 224. The receiver attenuator 224 further amplifies the signal by an amount determined by a digital attenuation signal received from the pulse sequence server 110. The received signal is at or around the Larmor frequency, and this high frequency signal is down converted in a two step process by a down converter 226. The down converter 226 first mixes the MR signal with the carrier signal on line 212 and then mixes the resulting difference signal with a reference signal on line 228 that is produced by a reference frequency generator 230. The down converted MR signal is applied to the input of an analog-to-digital (“A/D”) converter 232 that samples and digitizes the analog signal. The sampled and digitized signal is then applied to a digital detector and signal processor 234 that produces 16-bit in-phase (I) values and 16-bit quadrature (Q) values corresponding to the received signal. The resulting stream of digitized I and Q values of the received signal are output to the data acquisition server 112. In addition to generating the reference signal on line 228, the reference frequency generator 230 also generates a sampling signal on line 236 that is applied to the A/D converter 232.

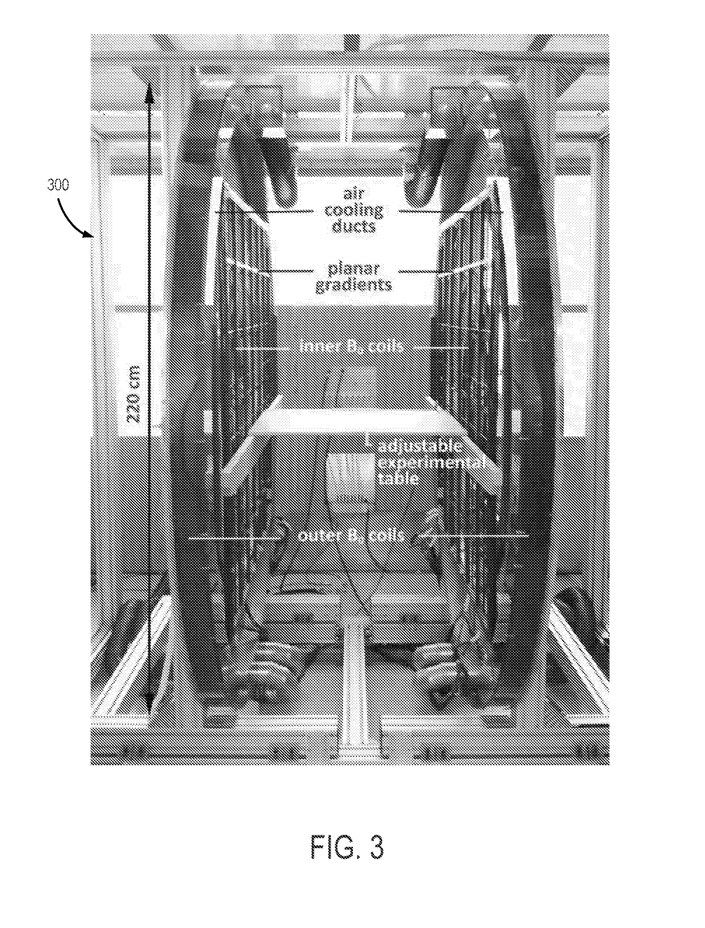

The basic MR systems and principles described above may be used to inform the design of other MR systems that share similar components but operate at very-different parameters. In one example, a low-field magnetic resonance imaging (lfMRI) system utilizes much of the above-described hardware, but has substantially reduced hardware requirements and a smaller hardware footprint. For example, referring to FIG. 3, a system 300 is illustrated that, instead of a 1.5 T or greater static magnetic field, utilizes a substantially smaller magnetic field. That is, in FIG. 3, as a non-limiting example, a 6.5 mT electromagnet-based scanner is illustrated. In particular, the system 300 includes a biplanar 6.5 mT electromagnet (B0) 302 that, for example, may be formed by inner B0 coils 304 and outer B0 coils 306. Biplanar gradients 308 may extend across the B0 electromagnet 302.

The system 300 may be tailored for ¹H imaging by achieving a high B0 stability, high gradient slew rates, and low overall noise. To achieve these ends, a power supply, for example, with +/−1 ppm stability over 20 min and +1-2 ppm stability over 8 h, may be used and high current shielded cables may be deployed throughout the system 300. In one non-limiting example, a power supply was adapted from a System 854T, produced by Danfysik, Taastrup, Denmark. The system 300 may operate inside a double-screened enclosure (ETS-Lindgren, St. Louis, Mo.) with a RF noise attenuation factor of 100 dB from 100 kHz to 1 GHz. In this example, the system may have a height, H, that is, as a non-limiting example, 220 cm. A cooling systems 310, such as may include air-cooling ducts, may be included.

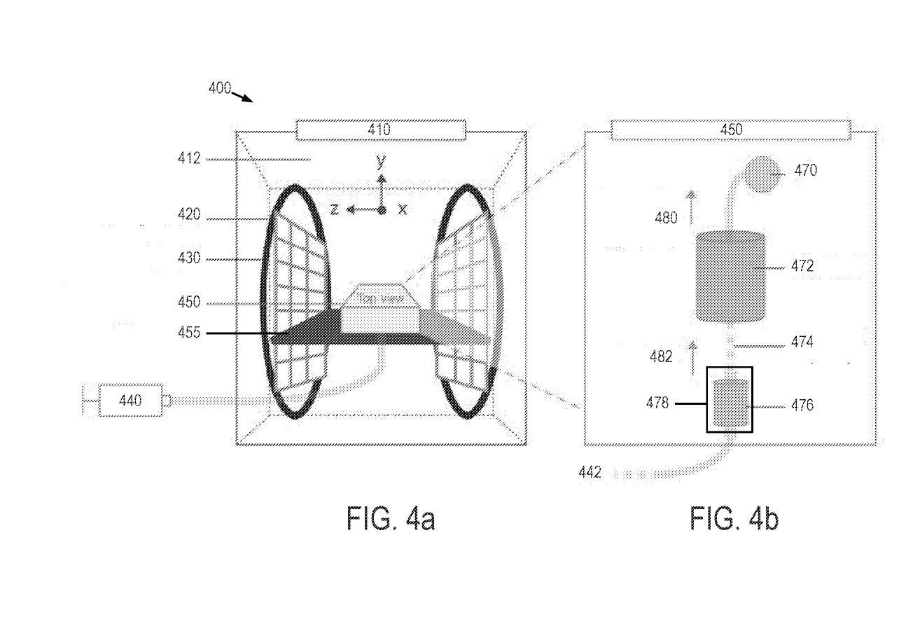

FIG. 4a is a block diagram of an MRI system in accordance with the present disclosure. In FIG. 4a, the MRI system 400 includes a main magnet system 410 configured to generate a main static magnetic field about at least a region of interest (ROI) of a subject arranged in the main magnet system. The main magnet system 410 may be a low filed MRI scanner that includes a low-field static magnetic field less than 10 mT. The main magnet system 410 includes a faraday cage 412, a planar gradient 420, a B0 coil 430, and a table 455. An sub-system 450 may be disposed on the table 455. The sub-system 450 is shown in a zoomed in view in FIG. 4b.

FIG. 4b is a zoomed in view of the MRI system in accordance with the present disclosure. As illustrated in FIG. 4b, the sub-system 450 includes an imaging coil 472, which may be configured to accommodate the ROI of the subject arranged in the main magnet system 410. The sub-system 450 includes a shielding box 478 that defines the at least partially shielded region in the shielding box 478. The sub-system 450 also includes a polarization magnet system 476 configured to generate a polarization static magnetic field in an at least partially shielded region away from the main magnet system 410. The polarization magnet system 476 is also configured to at least partially pre-polarize a blood substitute 442 to be injected to the subject.

The sub-system 450 receives fluid from a pump 440. As shown in FIGS. 4a-4b, the pump 440 is connected to one side of the polarization magnet system 476 through a first side of the shielding box 478, the pump configured to pump at least partially deuterated fluid 442 into the polarization magnet system 476. The fluid 442 may include blood substitute such as perfluoroctylbromide (PFOB), hemoglobin-based oxygen carriers (HBOC), perfluorocarbon-based oxygen carriers (PFBOC), or any other types of artificial blood substitute. The fluid 442 may be perflorinated before being pumped into the polarization magnet system 476. The fluid 442 may also include sterile saline.

The polarization magnet system 476 may be connected to the imaging coil 472 by a partially shielded capillary 474, which is connected to a second side of the polarization magnet system through a second side of the shielding box, the partially shielded capillary configured to transfer the pre-polarized blood substitute 482 to the subject. The output of the fluid 480 may be collected using a container 470. The partially shielded capillary may have a length of less than or equal to 1.0 m. Further, the polarization static magnet may be oriented 180° opposite to the main static magnet. Alternatively or additionally, the output of the fluid 480 may be circulated back to the polarization magnet system 476.

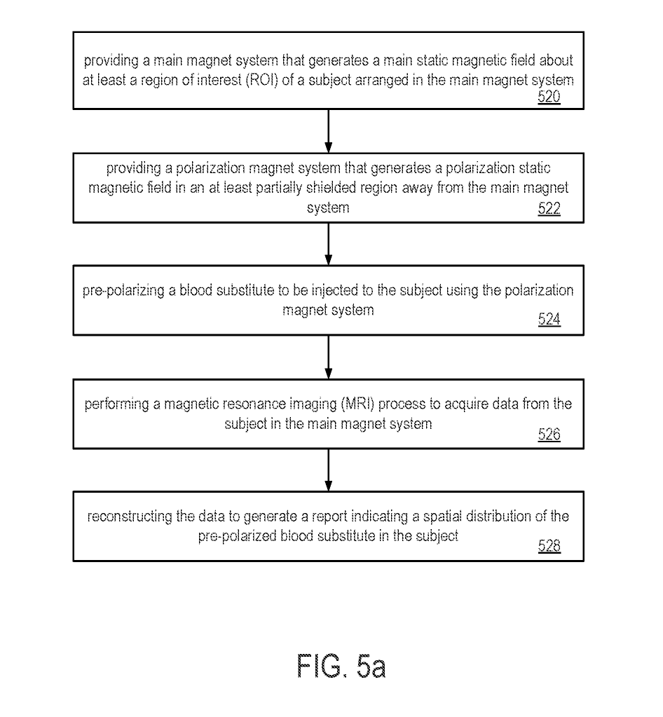

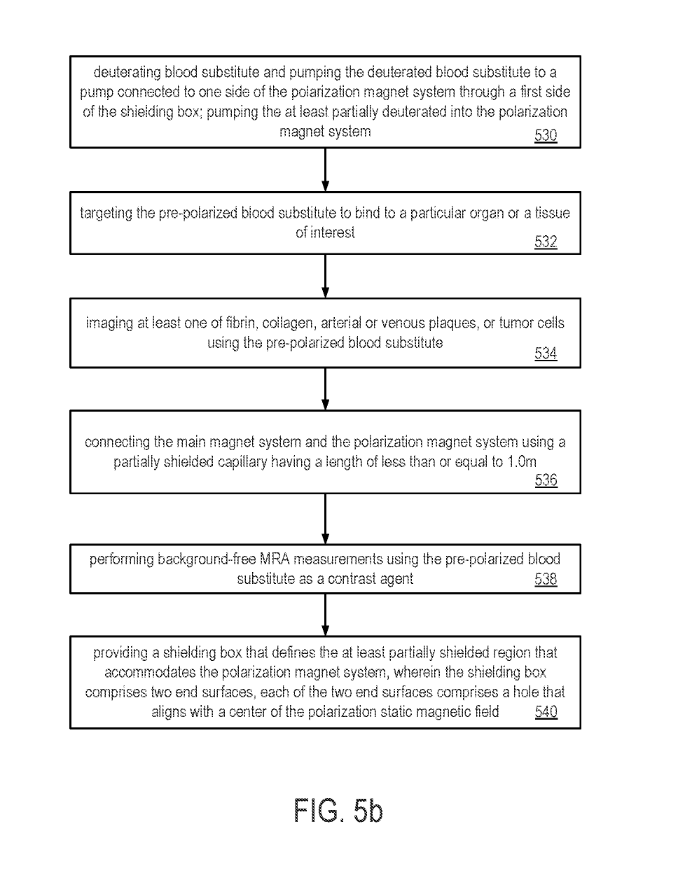

FIG. 5a illustrate an example method in accordance with the present disclosure. To perform a medical imaging process, the example method may be implemented in a MRI system that includes the following steps. In act 520, the method includes providing a main magnet system that generates a main static magnetic field about at least a region of interest (ROI) of a subject arranged in the main magnet system.

In act 522, the method includes providing a polarization magnet system that generates a polarization static magnetic field in an at least partially shielded region away from the main magnet system. The polarization magnet system may include a static magnetic field less than 1.5 T. For example, the polarization magnet system may include a small annular rare-earth magnet made from NdFeB with a magnetic field strength around 1.0 T. The relatively low field strength of the polarization magnet system makes it possible to place the polarization magnet system within 1.0 meter away from the main magnet system include a NMR coil. Accordingly, the partially shielded capillary connecting between the polarization magnet system and the main magnet system may have a length of less than or equal to 1.0 meter.

In act 524, the MRI system may pre-polarize a blood substitute to be injected to the subject using the polarization magnet system. Further, the polarization static magnet may be oriented 180° opposite to the main static magnet. Thus, the polarization magnet system may hyperpolarize the fluid in the opposite orientation. The signal (in the frame of the scanner magnet) caused by the hyperpolarized fluid starts out very large and negative, and then as it relaxed with T1 it would go through zero and the end up positive at the lower steady state thermal polarization of the main magnet system in the low field scanner.

In act 526, the MRI system may perform a MRI process to acquire data from the subject in the main magnet system. The subject may be a human, an animal, or a phantom. The MRI system may use different coils for different ROIs in the subject.

In act 528, the MRI system may reconstruct the data to generate a report indicating a spatial distribution of the pre-polarized blood substitute in the subject. For example, the report may indicate at least one of hyper-acute or mild blood brain barrier (BBB) disruption. The MRI system may use a workstation to reconstruct the images from the raw data and then generate the report on a display screen that communicates with the workstation.

FIG. 5b illustrate additional acts that may be implemented in accordance with the present disclosure. For example, in act 530, the MRI system may deuterate blood substitute and pump the deuterated blood substitute to a pump connected to one side of the polarization magnet system through a first side of the shielding box. The MRI system may pump the at least partially deuterated into the polarization magnet system.

In act 532, the MRI system may target the pre-polarized blood substitute to bind to a particular organ or a tissue of interest. In act 534, the MRI system may image at least one of fibrin, collagen, arterial or venous plaques, or tumor cells using the pre-polarized blood substitute. The MRI system may generate a report that indicates at least one of hyper-acute or mild blood brain barrier (BBB) disruption. In act 536, the MRI system may connect the main magnet system and the polarization magnet system using a partially shielded capillary having a length of less than or equal to 1.0 meter.

In act 538, the MRI system may perform background-free MRA measurements using the pre-polarized blood substitute as a contrast agent. For example, the MRI system may detect the fluorine resonance instead to imagine a perflorinated blood substitute like PFOB. In act 540, the MRI system may provide a shielding box that defines the at least partially shielded region that accommodates the polarization magnet system, where the shielding box includes two end surfaces, each of the two end surfaces comprises a hole that aligns with a center of the polarization static magnetic field.

For example, imaging may be performed at 0.0065 T using a low-field MRI scanner. The fluid hyper polarization may be performed using a 1.3 T permanent magnet placed in the Faraday cage, which is 1 meter away from the NMR coil. The permanent magnet may be a 5.5*5.5 cm2 cylinder with a 2 cm hole at its centre (K&J Magnetics, Pipersville, USA). The permanent magnet may be placed inside a custom-built shielding box after simulating the effect of the permanent magnet on B0 of our scanner using COMSOL Multiphysics (Burlington, USA).

To test the a low-field MRI scanner, a 10 mL modified plastic syringe containing fluid may be placed inside of the permanent magnet and may be connected on one side with a 60 mL syringe filled with fluid and placed on an infusion pump outside the Faraday cage, and with a PE50 capillary on the other side. The capillary may be in turn connected to a phantom, which include a modified 60 mL syringe allowing for continuous flow. A constant flow of 20 mL per minute may be started a few seconds before the acquisition began. A 17s-bSSFP sequence with 50% undersampling and 5 averages for a 2*2*10 mm³ spatial resolution may be used as a reference before repeating the same acquisition while injecting hyperpolarized water at a flow rate of 20 mL/min. The same scan with 20 averages may be used as a reference.

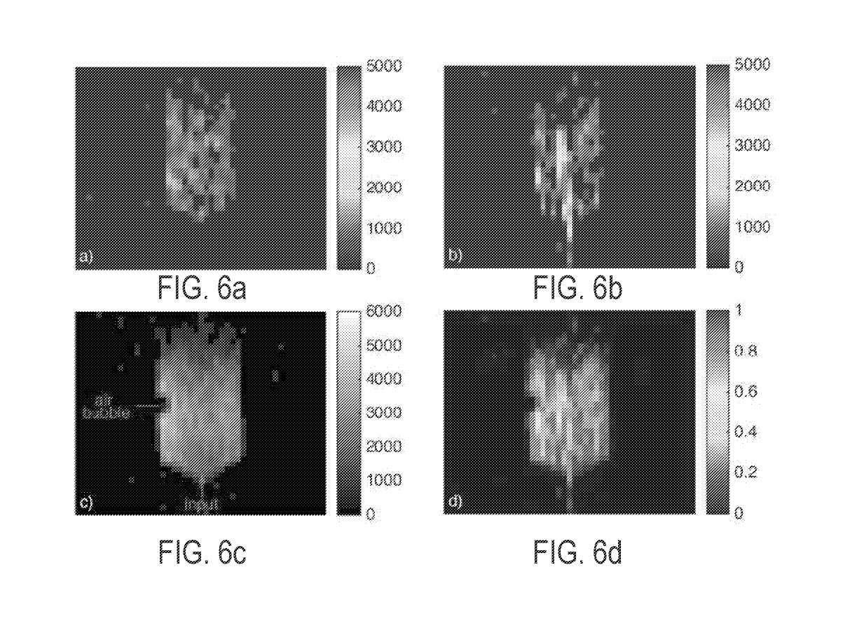

FIG. 6a shows a phantom image with 5 averages without hyperpolarized fluid. The phantom includes water without hyperpolarzed saline. The images may be reconstructed by the MRI workstation after performing the 17s-bSSFP sequence. The images may be processed using Matlab (Natick, USA) as well.

FIG. 6b shows a phantom image with 5 averages and hyperpolarized fluid. The hyperpolarized fluid may be input from a 1.3 T permanent magnet, and output to a waste container on the other side of the phantom.

FIG. 6c shows a reference scan with 20 averages where the input and output are clearly visible. The images may be reconstructed by the MRI workstation or using Matlab after performing the 17s-bSSFP sequence. The reconstructed images are then averaged to improve the signal to noise ratio.

FIG. 6d shows the overlay of FIG. 6b on FIG. 6c. The pixel value are normalized so that the pixel value is between 0 and 1.

The results in FIGS. 6a-6d show that the MRI system performs contrast-enhanced MR imaging at ultra-low magnetic field by using a strong permanent magnet to hyperpolarize fluid. The results show that the presence of a permanent magnet inside the Faraday cage affects T2* but not enough to prevent good quality imaging. A combination of stronger gradients and optimized NMR coils for receive would further improve the imaging quality, as well as an efficient way to inject hyperpolarized fluid in this setting with the help of the shielding box and the shielded capillary.

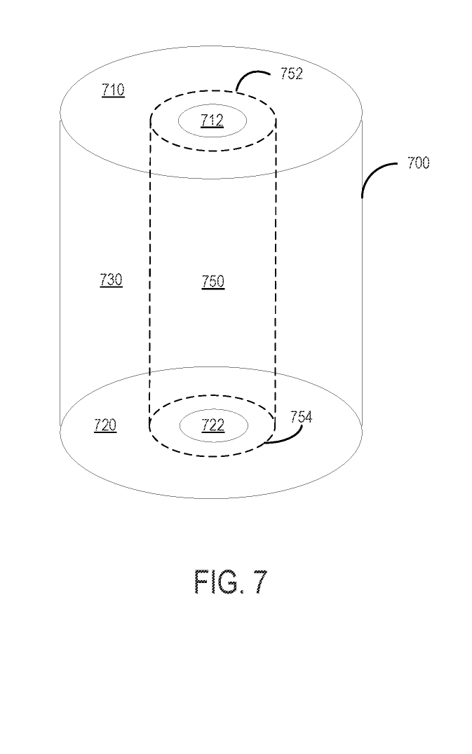

FIG. 7 shows a shielding box 700 according to the instant disclosure. The shielding box 700 includes a first end surface 710 and a second end surface 720. The first end surface 710 includes a hole to accommodate a pipe for transferring fluid such as a partially shielded capillary. The shielding box 700 may define the at least partially shielded region 730 that accommodates the polarization magnet system 750. The polarization magnet system 750 may generate a polarization static magnetic field inside the shielding box. Each of the two end surfaces 710 or 720 includes a hole 712 and 722. The holes may align with a center of the polarization static magnetic field generated by the polarization magnet system 750.

The magnetic shielding may be made of a square shape of iron that is 7 cm along each edge, and 1 mm thick on each side. The shielding box may include g-iron(mu=7000), a standard iron (mu=5000), or other type of material with a high mu value. Using the shielding box, the magnetic field of main magnet system is relatively homogeneous when the polarization magnet system is placed at about 1.0 meter from the main magnet system. The MRI system may further includes a controller that is configured to at least partially manipulate a strength of the pre-polarization by controlling a flow rate of the pre-polarized fluid.

Thus, a system and method is described for MRI that may be used in combination with an exogenously administered hyperpolarized fluid. As opposed to relaxation-based MRI contrast mechanisms, hyperpolarized fluid MRI signal enhancement can be modulated as desired by flow rate control of the hyperpolarized fluid to the main static magnet field, enabling contrast-enhanced MRI at a field strength less than 0.01 T. The present disclosure advantageously provides a non-invasive, fast operation and reduced SAR at low magnetic field with b-SSFP sequences.

The present disclosure has been described in terms of one or more embodiments, and it should be appreciated that many equivalents, alternatives, variations, and modifications, aside from those expressly stated, are possible and within the scope of the disclosure.

1. A magnetic resonance imaging (MRI) system, comprising:
a main magnet system configured to generate a main static magnetic field about at least a region of interest (ROI) of a subject arranged in the main magnet system;
at least one gradient coil configured to establish at least one magnetic gradient field with respect to the main static magnetic field;
a radio frequency (RF) system configured to deliver excitation pulses to the subject; and
a polarization magnet system configured to generate a polarization static magnetic field in an at least partially shielded region away from the main magnet system and at least partially pre-polarize a blood substitute to be injected to the subject.

2. The system of claim 1, further comprising:
a computer system programmed to control the at least one gradient coil and the RF system to perform a MRI pulse sequence;
wherein the computer system is further programmed to acquire data corresponding to signals from the subject having received at least partially pre-polarized blood substitute from the polarization magnet system.

3. The system of claim 2, wherein the computer system is further programmed to reconstruct, from the data, at least one anatomical image of the subject and spatially distributed pre-polarized blood substitute within the subject relative to the anatomical image.

4. The system of claim 1, wherein the main static magnetic field comprises a low-field static magnetic field less than 10 mT.

5. The system of claim 1, wherein the polarization static magnetic field comprises a static magnetic field less than 1.5 T.

6. The system of claim 1, further comprising a shielding box that defines the at least partially shielded region.

7. The system of claim 6, further comprising:
a pump connected to one side of the polarization magnet system through a first side of the shielding box, the pump configured to pump at least partially deuterated blood substitute into the polarization magnet system; and
a partially shielded capillary connected to a second side of the polarization magnet system through a second side of the shielding box, the partially shielded capillary configured to transfer the pre-polarized blood substitute to the subject.

8. The system of claim 7, wherein the partially shielded capillary has a length of less than or equal to 1.0 m.

9. The system of claim 1, wherein the polarization static magnet is oriented 180° opposite to the main static magnet.

10. A method for performing a medical imaging process, comprising:
providing a main magnet system that generates a main static magnetic field about at least a region of interest (ROI) of a subject arranged in the main magnet system;
providing a polarization magnet system that generates a polarization static magnetic field in an at least partially shielded region away from the main magnet system;
pre-polarizing a blood substitute to be injected to the subject using the polarization magnet system;
performing a magnetic resonance imaging (MRI) process to acquire data from the subject in the main magnet system; and
reconstructing the data to generate a report indicating a spatial distribution of the pre-polarized blood substitute in the subject.

11. The method of claim 9, further comprising targeting the pre-polarized blood substitute to bind to a particular organ or a tissue of interest.

12. The method of claim 11, further comprising imaging at least one of fibrin, collagen, arterial or venous plaques, or tumor cells using the pre-polarized blood substitute.

13. The method of claim 10, wherein the report indicates at least one of hyper-acute or mild blood brain barrier (BBB) disruption.

14. The method of claim 10, further comprising:
connecting the main magnet system and the polarization magnet system using a partially shielded capillary having a length of less than or equal to 1.0 m.

15. The method of claim 10, further comprising:
deuterating blood substitute and pumping the deuterated blood substitute to a pump connected to one side of the polarization magnet system through a first side of the shielding box; and
pumping the at least partially deuterated into the polarization magnet system.

16. The method of claim 10, further comprising performing background-free MRA measurements using the pre-polarized blood substitute as a contrast agent.

17. The method of claim 10, further comprising:
providing a shielding box that defines the at least partially shielded region that accommodates the polarization magnet system, wherein the shielding box comprises two end surfaces, each of the two end surfaces comprises a hole that aligns with a center of the polarization static magnetic field.

18. The method of claim 10, wherein the main static magnetic field comprises a low-field static magnetic field less than 10 mT.

19. The method of claim 10, wherein the polarization static magnetic field comprises a static magnetic field less than 1.5 T.

20. A magnetic resonance imaging (MRI) system, comprising:
a main magnet system configured to generate a main magnetic field about at least a region of interest (ROI) of a subject arranged in the MRI system;
a polarization magnet system configured to generate a polarization static magnetic field in an at least partially shielded region away from the main magnet system, the polarization magnet system configured to at least partially pre-polarize a fluid comprising fluorine;
a radio frequency (RF) system configured to deliver excitation pulses to the subject having received the pre-polarize a fluid comprising fluorine, wherein the excitation pulses comprises at least one embedded electron paramagnetic resonance (EPR) pulse; and
a controller configured to at least partially manipulate a strength of the pre-polarization by controlling a flow rate of the pre-polarized fluid.
